(19) United States
(12) Patent Application Publication
(10) Pub. No.: US 2012/0122237 A1
(43) Pub. Date: May 17, 2012

(54) HOMOGENEOUS NONCOMPETITIVE DETECTION OF POST TRANSLATIONAL MODIFICATIONS FOR USE IN HIGH THROUGHPUT ASSAYS

(76) Inventor: Robert G. Lowery, Belleville, WI (US)

(21) Appl. No.: 13/296,968

(22) Filed: Nov. 15, 2011

Related U.S. Application Data
(60) Provisional application No. 61/414,240, filed on Nov. 16, 2010.

(51) Int. Cl. G01N 33/53; C07K 2/00

(52) U.S. Cl. 436/501; 530/300

(57) ABSTRACT

A non-competitive immunoassay method for detection of post-translationally modified (PTM) proteins is disclosed. The method will enable the direct detection of PTM proteins in solution or on solid phase with a single reagent addition step, and with improved selectivity for specific PTM sites on target proteins. The key to the method is the use of a secondary binding molecule that specifically recognizes the immune complex between the primary antibody and the antigen.

The use of a secondary binding molecule will increase the sensitivity and selectivity of antigen detection compared to use of the primary antibody alone. The use of a secondary binding molecule also allows direct, homogenous detection rather than competition with a labeled tracer, because the secondary binding molecule can be labeled with a detection reagent that interacts with a detection reagent on the primary antibody.

### CROSS-REFERENCE TO RELATED APPLICATIONS

This present application is a Non-Provisional Patent Application which claims priority to U.S. Provisional Patent Application No. 61/414,240, filed Nov. 16, 2010, the disclosure of which is herein incorporated by reference in its entirety.

### STATEMENT REGARDING FEDERALLY SPONSORED RESEARCH OR DEVELOPMENT

This invention was made with United States government support under grant number R43 GM088945-01 awarded by the following government agency: National Institute of General Medical Sciences. The United States government has certain rights in this invention.

### BACKGROUND OF THE INVENTION

Specific detection of post translational modifications is a fundamental need in drug discovery and proteomics. Post translational modifications (PTMs) such as phosphorylation, glycosylation and methylation play a central and ubiquitous role in signal transduction at all levels in an organism [1]. The complexity of the proteome is increased by at least two orders of magnitude by covalent modifications [2], and the enzymes that catalyze these reactions comprise more than 5% of coding capacity of the human genome. PTMs control virtually every aspects of protein function, including localization, enzymatic activity, and interaction with other biomolecules in signaling networks, usually in a reversible manner. Not surprisingly, mapping PTM profiles in normal and diseased cells is a major goal of proteomics [3, 4] and PTM enzymes such as kinases and methyltransferases have become key targets for drug discovery. However, the fundamental analytic requirement for understanding how PTMs regulate cell function—detection of specific PTMs in complex mixtures—remains a daunting technical challenge for both fields [4-6].

The limited specificity of phosphoprotein antibodies necessitates a separation step, which limits their use in kinase drug discovery. Increasingly, new drug discovery efforts are shifting toward screening compound libraries in the more physiological context of intact cells instead of using biochemical assays with isolated proteins. The phosphorylation status of proteins is clearly a very attractive endpoint for cellular assays, both for screening kinases and for other targets that are linked to kinase signaling pathways [7]. The key technical challenge is how to detect specific changes in phosphorylation status in cells in a high throughput fashion. Typically cellular assays are run in 96- or 384-well plates, then cells are lysed or fixed, and antibodies are used to detect specific protein phosphorylation sites [8]. Though phosphoprotein antibodies are the most extensively developed, similar methods are used to detect specific methylation sites on proteins. Substantial progress has been made since the first phosphorylation state specific antibody was developed, however their ability to specifically recognize phosphorylation of a target protein at a single site still is limited [5, 9]. Even antibodies that are purported to be highly specific have been shown to cross-react with other sites after careful analysis [6]. For this reason, definitive detection of a specific phosphorylation event often requires a separation or enrichment step prior to immunodetection. The most common approach used for cell extracts is to resolve individual proteins using gel electrophoresis in one or two dimensions, then use immunoblotting with the phosphoprotein antibody [10, 11]. These methods are of limited utility from a drug discovery perspective because they cannot be easily integrated into an automated high throughput screening (HTS) workflow, which revolves around multiwell plates. Assays that require removal of samples from plates or that have outputs that cannot be read directly from plates are not commonly employed.

The lack of homogenous assay methods for phosphoprotein immunodetection further limits kinase drug discovery. Antibodies can be used for direct detection of PTMs in fixed cells in cases where specificity is adequate or not critical, however the complexity of the immunocytochemical protocols used complicates their incorporation into HTS. For ease of automation, HTS campaigns almost always rely on a homogenous assay; i.e., one that can be run without multiple liquid addition and removal steps. Although it is technically feasible, the logistics of incorporating a full immunocytochemical protocol into a primary screen of any appreciable size are intimidating [8]. This is unfortunate, as it means that the immediate effect of protein kinases is seldom used as an output in a primary screen. Indirect assays for downstream effects such as gene expression are used as more HTS-friendly alternatives, but they are much less specific than direct measurement of target protein phosphorylation. Even when analysis of phosphorylation patterns is used as a secondary screen, the scope of the analysis is often limited by the cumbersome nature of the immunocytochemistry protocol. Thus the lack of homogenous assay methods for PTMs is seriously hampering drug discovery for kinases and emerging PTM families such as methyltransferases. Though there are isolated cases of HTS-compatible methods for PTM detection [12-14], there is no broadly applicable approach that overcomes both the specificity and homogenous detection problems.

The need for a new assay method for the detection of PTMs will benefit the process of drug discovery. As we have described, an entirely novel method for the detection of PTMs is necessary to move the field of drug discovery forward. These small changes in the structure of proteins regulate their activity and play a key role in determining their involvement in disease processes. Methods for detecting these changes have not kept pace with the scale necessary within the current pharmaceutical discovery climate. The invention described in the instant application overcomes many of the limitations of the current detection systems by describing an entirely new way of recognizing these small changes in a format which can be adopted by the pharmaceutical industry.

### BRIEF SUMMARY OF THE INVENTION

This invention encompasses methods and reagents for detection of PTMs in complex mixtures in a single step, homogenous assay format that is well suited for use in an automated, high throughput mode. Specifically, the invention involves the application of a non-competitive immunoassay method—originally developed for detection of small antigens such as pesticide residues—for detection of phosphorylated proteins. The invention will enable the direct detection of phosphorylated proteins in solution or on solid phase with a single reagent addition step, and with improved selectivity for specific phosphorylated sites on target proteins. The ability to directly detect phosphoproteins in cell extracts without fixation or wash steps would greatly streamline a very common assay used in drug discovery and research settings, and thus would have significant commercial potential.

The key to the method is the use of a secondary binding molecule that specifically recognizes the immune complex between the primary antibody and the antigen. The use of a secondary binding molecule has been shown to increase the sensitivity and selectivity of antigen detection compared to use of the primary antibody alone. The use of a secondary binding molecule also allows direct, homogenous detection rather than competition with a labeled tracer because the secondary binding molecule can be labeled with a detection reagent that interacts with a detection reagent on the primary antibody. For instance, the primary antibody can be labeled with a FRET donor, and the secondary binding molecule can be labeled with an acceptor so that a fluorescence signal is generated only when the complete trimolecular complex between antibody, antigen and secondary binding molecule is formed.

The case of phosphoproteins and other modified proteins is somewhat unique because whereas the antigen being detected is a large molecule, only a small portion of it is generally used as immunogen, typically the phosporylated or otherwise modified amino acid and a few flanking amino acids. Thus, the primary antibody can be produced using a small phosphopeptide or appropriately modified peptide and the secondary binding molecule can be produced using the complex between the primary antibody and the phosphopeptide or appropriately modified peptide, but in practicing the invention the reagents would be used to detect the much larger intact phosphorylated or otherwise modified protein.

The secondary binding molecule can be a native polyclonal or monoclonal antibody, however the isolation of such anti-immune complex antibodies has historically been very difficult. Recent improvements have been the use of recombinant antibodies or even short peptides, both selected from phage display libraries (15).

### BRIEF DESCRIPTION OF THE DRAWINGS

FIG. 1 is a model of the HT-PTM assay system of the current invention. This assay system can be used for the homogeneous detection of modified proteins (like phosphorylations or methylations). Peptides with attached terbium (Tb) labels which specifically recognize antibody-modified protein complex (phosphorylated in the model shown in FIG. 1) including parts of both molecules, bind to form a trivalent immune complex. This trivalent conformation relieves the quenching of the attached Tb and results in the generation of a time resolved fluorescence signal.

FIG. 2 is a comparison of the experimental steps involved for the utilization of the current invention compared with the existing technology of immunocytochemistry for the detection of post translational modifications.

### DETAILED DESCRIPTION OF THE INVENTION

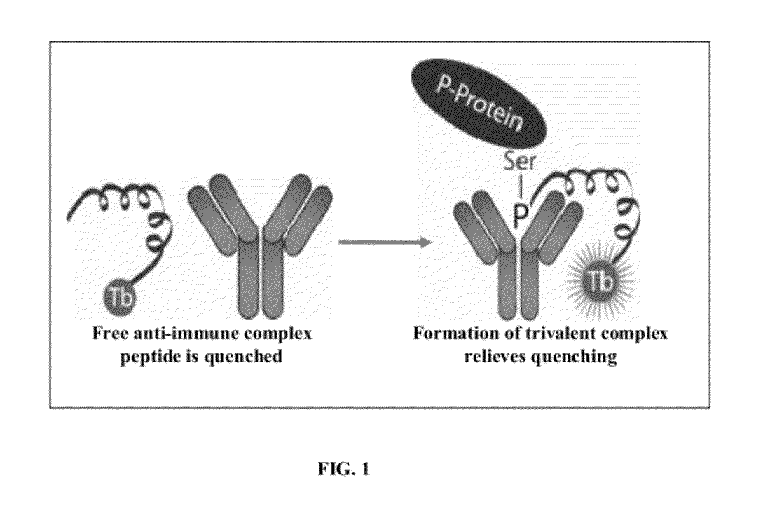

The HT-PTM assay described here will overcome the key technical hurdles to HTS cellular assays for phosphoprotein detection. In the invention described in the instant application, the HT-PTM assay offers the increased specificity needed to eliminate a separation step prior to immunodetection of PTMs. This specificity is imparted by anti-immune complex (anti-IC) peptides that specifically recognize and bind to antibody-phosphoprotein complexes (FIG. 1).

The antibody-antigen-peptide trivalent immune complex approach, also known as Phage Anti-Immune Complex (PHAIA) because it relies on phage display peptide libraries, has been shown to reduce the cross-reactivity of both polyclonal and monoclonal antibodies by more than 100-fold (Table 1) [15, 16] in other antigen detection systems. In addition, this approach has increased the sensitivity of antigen detection by more than 10-fold. Assays are designed such that signal generation is dependent on the peptide binding at the interface of the antibody-antigen complex—apparently recognizing parts of both molecules (see FIG. 1). Thus the peptide imparts another level of specificity that is dependent on the structure of the epitope and the antibody in complex.

TABLE 1
Crossreactivity comparison using competitive ELISA and PHAIA.
(data from publications 15 and 16)

Crossreactivity (%)

| | | |
|---|---|---|
| Compound | ELISA | PHAIA |

Monoclonal Antibody for Atrazine

| | | |
|---|---|---|
| atrazine | 100 | 100 |
| simazine | 48 | 0 |
| propazine | 116 | 144 |
| cyanazine | 91 | 0 |

Polyclonal antibody for 3-PBA

| | | |
|---|---|---|
| 3-PBA | 100 | 100 |
| 3-pheonoxy aldehyde | 10 | 3 |
| 4-hydroxy 3-PBA | 98 | 0 |

The instant application defines a system for looking at extremely small changes to large molecules. Prior patents from the current inventors (U.S. Pat. Nos. 7,332,278, 7,355,010 and 7,378,505) have demonstrated what many believed was impossible—the detection of differences by antibodies of extremely small differences between small molecules. This prior art has shown that extremely specific antibodies can be developed which recognize the difference between molecules which differ by only a single phosphate group.

This prior experience by the inventors led them to a novel idea; perhaps peptides could be selected from a phage display library which have the exquisite specificity to bind to an immune complex (like PHAIA) made up of an antibody and a very particular large molecule. This large molecule is present in two different states—phosphorylated and non-phosphorylated. These states differ only by one small phosphate group, and the peptide has the ability to recognize the difference between these two states when bound to an antibody, thereby increasing sensitivity and specificity.

With the instant invention, scientists will be able to detect direct and highly specific cellular phosphorylation events in a homogenous format (lyse cells, add detection reagents, read plates) using existing phosphoprotein antibodies.

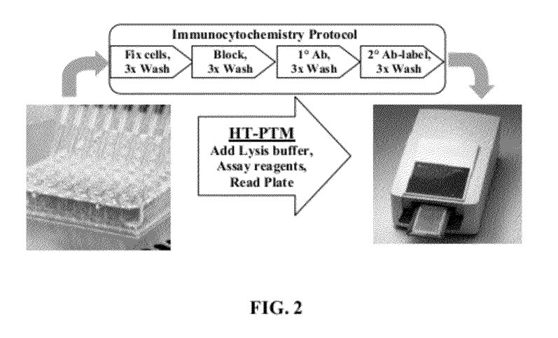

The workflow for the HT-PTM compared to existing immunocytochemistry and Western blot protocols for phosphoprotein detection are shown in FIG. 2. The advantages of over current methods include:
Increased specificity and sensitivity for phosphoprotein detection: overcomes antibody specificity limitations, eliminating the need for a separation step (e.g., gel electrophoresis).
Homogenous detection of P-proteins in cell extracts: eliminates need the for immunocytochemistry protocols involving fixation and multiple incubation/wash steps.
Broadly applicable because it builds on a vast repertoire of existing P-protein antibodies: new assays can be developed using a rapid, in vitro panning process for identification of anti-IC peptides.

The availability of cellular assays for specific phosphorylation events would have a broad and significant impact on drug discovery in diverse therapeutic areas. The HT-PTM overcomes the key technical hurdles preventing the development of HTS cellular assays for measuring specific protein phosphorylation events. Its use would allow large scale screening and profiling of kinase inhibitors in the physiological context of intact cells with the most immediate effect of kinase activity as the endpoint. This capability could result in the identification of new classes of kinase inhibitors with higher and/or more defined specificity and different mechanisms of action than current kinase drugs, which mostly resulted from screens using biochemical assays. The potential medical impact is extremely high, since kinases have very diverse disease links and are actively being pursued as targets in almost every therapeutic area, including oncology, inflammatory disorders, anti-infectives and metabolic disorders [3, 17-21]. Moreover, specific phosphorylation events can be used as surrogate endpoints for other target classes such as GPCRs [7], broadening the impact of the HT-PTM assay even further.

In addition to the impact on drug discovery, the specificity enhancement provided by the HT-PTM assay could benefit proteomics efforts to map PTMs in tissue samples. Antibody crossreactivity is acknowledged as a key uncertainty in histochemical phosphoprotein profiling efforts [5]. The anti-IC peptides (when left attached to phage particles) can be used with common histochemical detection methods such as ELISA [15, 16]. Thus the technology has the potential to enhance efforts to understand how PTMs regulate cell function in normal and diseased tissues.

Sensitive cellular assays for phosphorylation events by HT-PTM offers significant innovation over currently available techniques. The HT-PTM assay that we propose to develop is innovative primarily because it overcomes two longstanding technical hurdles preventing the use of specific phosphorylation events as endpoints in cellular HTS assays, and in doing so it sets the stage for significant advances in kinase drug discovery and proteomics.

The fact that the HT-PTM improves upon the specificity of existing PTM antibodies also is innovative because it leverages the enormous amount of effort that has already gone into their development and will allow rapid introduction of new assays.

A variety of detection methods could be utilized with the HT-PTM method.

QRET. Homogenous detection in the HT-PTM assay could be provided by quenched resonance energy transfer (QRET), which reports on binding of the anti-IC peptide to the antibody-phosphoprotein complex. QRET employs a proprietary soluble leucoberbelin blue I quencher and a chelated lanthanide ion, such as terbium or europium [22-24]. The lanthanide fluorescence is efficiently quenched when attached to a small molecule free in solution, and quenching is relieved when it binds to a macromolecule. QRET provides the high sensitivity and time gated aspects of lanthanide fluorescence in a single label format. It has been demonstrated thus far in an immunoassay for estradiol [23], a GTP-Gα protein binding assay [24], and a GPCR receptor-ligand binding assay [17], which was used to correctly identify β-adrenergic receptor ligands. The use of QRET as a detection method is innovative, not just because it is new, but because it has significant advantages for the HT-PTM assay that would not be available with other detection modes.

TR-FRET. Time-resolved Forster-resonance-energy-transfer (TR-FRET) could be used as a competitive assay for peptide binding to the immune complex with a far-red TR-FRET readout. It would be used for HTS with a single addition mix-and-read format, reagent stability, and compatibility with commonly used multimode plate readers. The red tracer could be bound to the peptide molecule and terbium bound to the anti-phosphoprotein antibody. When the peptide is bound to the complex excitation of the terbium with UV light results in energy transfer to the tracer on the peptide and an increase in FRET with a time delay.

FP. Fluorescence polarization (FP) is a commonly used tool for investigating molecular processes which involve interactions or fluidity. FP is a homogeneous technology and reactions are very rapid, taking seconds to minutes to reach equilibrium. Because FP is a fundamental property of the molecule, the reagents are stable, and this results in a high level of reproducibility. FP has proven to be highly automatable, often performed with a single incubation and a single, premixed, tracer-receptor reagent. The fact that there are no washing steps increases the precision and speed over heterogeneous technologies and dramatically reduces waste. FP is independent of fluorescence intensity, so this technology is unaffected by turbidity or the presence of dyes. This means that this detection method may be well suited to usage with the cell lysates described in the instant invention. High-throughput screening is now accommodated with the advent of polarizing optics on microplate readers.

FP is a technique perfectly aligned with the study molecular interactions. It gives a direct, nearly instantaneous measure of a tracer's bound/free ratio. FP experiments are done in solution, allowing true equilibrium analysis down to the low picomolar range. FP measurements do not adulterate samples, so they can be treated and reanalyzed in order to ascertain the effect on binding by changes such as pH, temperature, and salt concentration.

FP measurements are based upon the assessment of the rotational motions of a molecule. It is extremely efficient at measuring discreet differences in molecular mass which occur during large molecule/small molecule interactions, such as antibody/ligand or in the case of the instant invention, the binding of the PHAIA peptide (small) to the antibody/phosphorylated protein complex (very large). In general, a dye is attached to a small molecule, or tracer. It is then possible to measure its binding to a molecule of greater size, through its speed of rotation, in real-time or as an end-point assay.

When a tracer molecule (fluorophore bound to peptide molecule) is excited by polarized light, it will emit light in the same polarized plane, provided that the molecule remains stationary throughout the excited state. If the molecule rotates and tumbles out of this plane during the excited state, light is emitted in a different plane from the excitation light. If vertically polarized light is exciting the fluorophore, the intensity of the emitted light can be monitored in vertical and horizontal planes (degree of movement of emission intensity from vertical to horizontal plane is related to the mobility of the fluorescently labeled molecule). If a molecule is very large, little movement occurs during excitation and the emitted light remains highly polarized. If a molecule is small, rotation and tumbling is faster and the emitted light is depolarized relative to the excitation plane. Large molecules tend to rotate slowly while small molecules rotate rapidly, as the rate of rotation is inversely proportional to a molecule's size. Small molecules rotate quickly during the excited state, and upon emission, have low polarization values. Large molecules, caused by binding of a second molecule, rotate little during the excited state, and therefore have high polarization values. This change in rotational speed is the essence of all FP assays.

### Definitions:

As used herein, the term “Post translational modifications” or “PTMs” refers to any of a series of changes which can occur to a protein molecule after it is initially produced by translation. Non-limiting examples of such changes include phosphorylation by protein kinases or methylation by methyltransferases. These changes may or may not affect the structure or function of the protein molecule, but are often key changes in the regulation of protein activity.

The term “High throughput” of “HT” as used herein refers to the ability to accomplish testing quickly and efficiently on large numbers of samples.

The term “High throughput screening” or “HTS” as used herein refers to the testing within a short time period of many thousands of molecules (or test compounds) for their effects on changes in function and/or post translational modifications of a protein. Related screening methods are known in the art and they are generally performed in multiwall plates with automated liquid handling and detection equipment; however, it is imagined that the methods of the invention may be practiced on a microarray or in a microfluidic system.

The term “Phage display” as used herein refers to a method for screening large numbers of peptides for their ability to bind to a specific entity (an immune complex in the case of the instant invention). A library of peptide molecules are displayed on the surface of viral particle and allowed to bind with the immobilized target of interest. Unbound peptide/phage is washed away. Bound peptide/phage can be amplified and used to produce the peptide of interest.

The term “Phage display anti-immune complex peptides” or “PHAIA-derived peptide” as used herein refers to any of a series of peptides isolated using the process of phage display.

The term “Quenched resonance energy transfer” or “QRET” as used herein refers to a homogeneous detection method for the trivalent immune complex using a single label which is bound to the peptide molecule. Upon binding of the peptide to the antibody-phosphoprotein complex to form the trivalent immune complex the attached lanthanide label is relieved of quenching and results in the generation of a time resolved fluorescence signal. QRET employs a soluble leucoberbelin blue I quencher to achieve quenching when the peptide is unbound.

The term “Time-resolved Forster-resonance-energy-transfer” or “TR-FRET” as used herein refers to a method used to detect molecular interaction by measure an energy transfer between a fluorescent tracer molecule and a lanthanide (terbium) complex. Excitation of the terbium with UV light results in an energy transfer to the tracer molecule and emission at a higher wavelength after a time delay. The emission can be detected and reflects the level of binding of the molecules containing the lanthanide complex and the fluorescent tracer molecule.

The term “Fluorescent polarization” or “FP” as used herein refers to a method which can be used to detect molecular interactions by measuring a change in the planes of emission of polarized light. The rotational speed within the emission plane for a given molecule is inversely proportional to a molecule's size.

### Methods and Materials:

The HT-PTM assay enables direct and highly specific detection of cellular phosphorylation events in a homogenous format (lyse cells, add detection reagents, read plates) using existing phosphoprotein antibodies. The workflow for the HT-PTM compared to existing immunocytochemistry and Western blot protocols for phosphoprotein detection are shown in FIG. 2. The advantages of over current methods include:
Increased specificity and sensitivity for phosphoprotein detection: overcomes antibody specificity limitations, eliminating the need for a separation step (e.g., gel electrophoresis).
Homogenous detection of P-proteins in cell extracts: eliminates need the for immunocytochemistry protocols involving fixation and multiple incubation/wash steps.
Broadly applicable because it builds on a vast repertoire of existing P-protein antibodies: new assays can be developed using a rapid, in vitro panning process for identification of anti-IC peptides.
Detection of binding of the specific peptide could be accomplished with a variety of technology formats. QRET detection provides extremely sensitive, time resolved detection with a positive signal. In addition, a competitive binding method like TR-FRET could be utilized. Fluorescence polarization is another possibility where binding of the peptide within the large antibody-antigen-peptide trivalent immune complex would radically change the polarization pattern seen with a suitable tracer molecule.

Panning phage display libraries for anti-IC peptides. To identify candidate anti-IC peptides, phage display libraries are panned for peptides that bind specifically to antibody-phosphopeptide immune complexes.

To reduce the amount of antibody required (and thus the cost), we use streptavidin coated magnetic bead as the solid phase rather than coating wells with antibody, an approach recently shown to reduce the amount of antibody required for PHAIA detection by 10-fold relative to coated wells [25]. Antibodies are labeled via amino groups with biotin (EZ-Link sulfo-NHS-LC-Biotin, Pierce, Rockford, Ill.) and bound to strepatavidin coated magnetic beads (Invitrogen, Carlsbad, Calif.) at a high density. For biopanning, we use M13 phage display libraries comprised of random cyclic 7- to 10-mers with the general sequence ASGSACX₈CGP_{6—}, linked to the N-terminus of the major pVIII protein via a flexible linker (GG-C(X)₇₋₁₁C(GGGGS)₃₋) in the phagemid vector pAFF/MPB (a gift from Affymax Research Institute, Palo Alto, Calif.) with an estimated diversity of 3×10¹⁰ independent clones. E. coli ARI 292 cells (Affymax Research Institute) are used as the host strain. Beads coated with phosphoprotein antibodies are incubated in 96 well plates with phage preps in the presence of saturating concentrations of the target phosphopeptide; i.e., antibody-phosphopeptide immune complexes should be the predominant species displayed on the beads. Care is taken to prevent dephosphorylation of phosphopeptide antigens by testing for phosphatase activity in phage suspensions and adding inhibitors such as sodium vanadate, if necessary. After washing to remove non-specifically bound phage particles, anti-IC peptides are detected by bead-based ELISA using anti-M13 phage coat antibody conjugated to horseradish peroxidase (pAbM13-HRP) as secondary antibody (GE Health Care, Piscataway, N.J.). After 3-4 cycles of panning and phage amplification, the phages expressing anti-IC specific peptides are tested for detection of the desired phosphoprotein.

Detection of anti-immune complex peptide binding in a trivalent complex with QRET technology. For the QRET assay, anti-IC peptides that result in highly specific phosphoprotein detection are synthesized with Eu(III) and Tb(III) isothiocyanate chelates (PerkinElmer) attached at the end of short, flexible linkers (GGS) at the C-terminus (where the peptides were attached to the phage coat protein). The QRET technique relies on “protection” of the lanthanide by macromolecules to relieve quenching, thus the tracer must remain relatively small.

Tracers can then be quenched with leucoberbelin blue I quencher, and antibodies and phosphopeptides are titrated in a checkerboard fashion to determine the optimal concentrations for various detection ranges, and the specificity of the QRET-based HT-PTM is confirmed by testing crossreaction with secondary phosphopeptides. Effects of reagents used for cell lysis (e.g., Triton X-100) are tested to insure compatibility and/or identify the optimal method. All assay development is carried out in 384 well plates, and the QRET time resolved fluorescence signals will be read on a Tecan Safire II plate reader with appropriate filters and time delays for the Eu and Tb lanthanides tested.

### Examples

The HT-PTM assay enables the detection of a variety of post translational modifications in a homogeneous format directly from cell lysates. The HT-PTM assay enables the detection in cell lysates of phosphorylation changes and utilizes existing commercially available phosphoprotein antibodies. The HT-PTM assay also enables the detection of protein methylation sites, since methyltransferases are increasingly being targeted in oncology and technical hurdles to HTS detection of these changes are similar to those seen with phosphorylation.

The HT-PTM assay enables pharmacologic screening for kinase inhibitors in a physiological context. The HT-PTM overcomes the key technical hurdles preventing the development of HTS cellular assays for measuring specific protein phosphorylation events. Its use allows large scale screening and profiling of kinase inhibitors in the physiological context of intact cells with the most immediate effect of kinase activity as the endpoint. For example, cells derived from a tumor could be grown in multiwell plates, activated for proliferation with a growth factor such as EGF which is known to effect changes in cellular phosphorylation and treated with various test compounds. Cells could then be lysed in the wells, the HT-PTM reagents could be added, and the phosphoylation status of a specific protein could be ascertained by measuring the signal from the HT-PTM reagents using a plate reader. In this way, compounds which have effects on the phosporylation of the specific target protein could be identified. This capability could result in the identification of new classes of kinase inhibitors with higher and/or more defined specificity and different mechanisms of action than current kinase drugs, which mostly resulted from screens using biochemical assays. The potential medical impact is extremely high, since kinases have very diverse disease links and are actively being pursued as targets in almost every therapeutic area, including oncology, inflammatory disorders, anti-infectives and metabolic disorders [3, 17-21]. Moreover, specific phosphorylation events can be used as surrogate endpoints for other target classes such as GPCRs [7], broadening the impact of the HT-PTM assay even further.

In an additional drug discovery application similar yet distinct from the above, the HT-PTM could be used to measure protein kinase activity in extracts of cells. In one use of this example, the cells would be first cultured, then treated with an effector molecule, then lysed, and the activity of the target kinase could be measured in the cellular extract. This would be useful for kinase regulatory systems that cannot be recapitulated with purified components, or for test compounds that cannot efficiently pass through the cell membrane. Another use of this application would be to monitor the activity of kinases in patient samples to assess the effects of an investigational drug that is a kinase inhibitor or is expected to affect a kinase pathway indirectly.

In addition to drug discovery, the HT-PTM assay enables the mapping of post translational modifications in tissue samples. Antibody crossreactivity is acknowledged as a key uncertainty in histochemical phosphoprotein profiling efforts [5]. Though the QRET element would not likely be useful for this application, the anti-IC peptides (when left attached to phage particles) can be used with common histochemical detection methods such as ELISA [15, 16]. Thus the technology has the potential to enhance efforts to understand how PTMs regulate cell function in normal and diseased tissues.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Multiple embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein.

Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All publications and patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

### RELATED PUBLICATIONS

1. Walsh, C. T., S. Garneau-Tsodikova, and G. J. Gatto, Jr., Protein posttranslational modifications: the chemistry of proteome diversifications. Angew Chem Int Ed Engl, 2005. 44(45): p. 7342-72.
2. Yang, X. J., Multisite protein modification and intramolecular signaling. Oncogene, 2005. 24(10): p. 1653-62.
3. Ashman, K. and E. L. Villar, Phosphoproteomics and cancer research. Clin Transl Oncol, 2009. 11(6): p. 356-62.
4. Teraishi, T. and K. Miura, Toward an in situ phospho-protein atlas: phospho- and site-specific antibody-based spatio-temporally systematized detection of phosphorylated proteins in vivo. Bioessays, 2009. 31(8): p. 831-42.
5. Mandell, J. W., Immunohistochemical assessment of protein phosphorylation state: the dream and the reality. Histochem Cell Biol, 2008. 130(3): p. 465-71.
6. Gallo, R. M. and D. J. Riese, 2nd, The antibody sc-33040-R fails to specifically recognize phosphorylation of ErbB4 on tyrosine1056. Growth Factors, 2007. 25(5): p. 329-33.
7. Crouch, M. F. and R. I. Osmond, New Strategies in Drug Discovery for GPCRs: High Throughput Detection of Cellular ERK Phosphorylation. Comb Chem High Throughput Screen, 2008. 11(5): p. 344-56.
8. Garton, A. J., L. Castaldo, and J. A. Pachter, Quantitative high-throughput cell-based assays for inhibitors of ROCK kinases. Methods Enzymol, 2008. 439: p. 491-500.
9. Waraich, R. S., et al., Development and precise characterization of phospho-site-specific antibody of Ser(357) of IRS-1: elimination of cross reactivity with adjacent Ser(358). Biochem Biophys Res Commun, 2008. 376(1): p. 26-31.
10. Robinson, A. A., et al., Protective effect of phosphorylated Hsp27 in coronary arteries through actin stabilization. J Mol Cell Cardiol.
11. Sawasdikosol, S., Detecting tyrosine-phosphorylated proteins by Western blot analysis. Curr Protoc Immunol. 3: Chapter 11: p. 1-11.
12. Carlson, C. B., et al., Development of LanthaScreen cellular assays for key components within the PI3K/AKT/mTOR pathway. J Biomol Screen, 2009. 14(2): p. 121-32.
13. Dudek, J. M. and R. A. Horton, TR-FRET biochemical assays for detecting posttranslational modifications of p53. J Biomol Screen, 2009. 15(5): p. 569-75.
14. Kong, A., et al., Prognostic value of an activation state marker for epidermal growth factor receptor in tissue microarrays of head and neck cancer. Cancer Res, 2006. 66(5): p. 2834-43.
15. Gonzalez-Techera, A., et al., Phage anti-immune complex assay: general strategy for noncompetitive immunodetection of small molecules. Anal Chem, 2007. 79(20): p. 7799-806.
16. Gonzalez-Techera, A., et al., Polyclonal antibody-based noncompetitive immunoassay for small analytes developed with short peptide loops isolated from phage libraries. Anal Chem, 2007. 79(23): p. 9191-6.
17. Adams, J. L., et al., p38 MAP kinase: molecular target for the inhibition of pro-inflammatory cytokines. Prog Med Chem, 2001. 38: p. 1-60.
18. Bullock, W. H., et al., Prospects for kinase activity modulators in the treatment of diabetes and diabetic complications. Curr Top Med Chem, 2002. 2(9): p. 915-38.
19. Burgess, S. and V. Echeverria, Raf inhibitors as therapeutic agents against neurodegenerative diseases. CNS Neurol Disord Drug Targets. 9(1): p. 120-7.
20. Cohen, P., Protein kinases—the major drug targets of the twenty-first century? Nat Rev Drug Discov, 2002. 1(4): p. 309-15.
21. Dalrymple, S. A., p38 mitogen activated protein kinase as a therapeutic target for Alzheimer's disease. J Mol Neurosci, 2002. 19(3): p. 295-9.
22. Harma, H., et al., A new simple cell-based homogeneous time-resolved fluorescence QRET technique for receptor-ligand interaction screening. J Biomol Screen, 2009. 14(8): p. 936-43.
23. Harma, H., et al., Comparison of homogeneous single-label fluorometric binding assays: fluorescence polarization and dual-parametric quenching resonance energy transfer technique. Anal Chem. 82(3): p. 892-7.
24. Rozwandowicz-Jansen, A., et al., Homogeneous GTP binding assay employing QRET technology. J Biomol Screen. 15(3): p. 261-7.
25. Kim, H. J., et al., Magnetic bead-based phage anti-immunocomplex assay (PHAIA) for the detection of the urinary biomarker 3-phenoxybenzoic acid to assess human exposure to pyrethroid insecticides. Anal Biochem, 2009. 386(1): p. 45-52.

We claim:

1. A method for the detection of a small molecular change to a large molecule comprising the steps of:
(a) contacting the large molecule with a specific binding partner to form a complex,
(b) contacting the complex with a peptide to form a trimolecular complex, and
(c) detecting the binding of the peptide to the complex at the site of the small molecular change, thereby detecting the small molecular change.

2. The method of claim 1, wherein the step of detecting the binding of the peptide to the complex takes place in a homogeneous assay.

3. The method of claim 1 wherein the small molecular change to a large molecule that is detected is a post translational modification (PTM) of a protein.

4. The method of claim 3, wherein the post translational modification is selected from the group consisting of phosphorylation, methylation, glycosylation, acetylation, ADP-ribosylation, nitrosylation, or farnesylation.

5. The method of claim 3, wherein the peptide enhances the specificity of the PTM detection relative to the specificity observed with the specific binding partner alone.

6. The method of claim 1, wherein the specific binding partner is an antibody or part of an antibody molecule.

7. The method of claim 1, wherein the peptide is derived from a phage display library.

8. The method of claim 1 wherein the step of detecting the binding of the peptide to the complex is performed by quenched resonance electron transfer (QRET), fluorescence polarization (FP) or time resolved Forster resonance energy transfer (TR-FRET).

9. A peptide used for the detecting small molecular changes to a large molecule, the peptide being capable of binding to a complex formed by the combination of a large molecule and a specific binding partner.

10. The peptide of claim 9, wherein the large molecule is a post translationally modified protein.

11. The peptide of claim 10, wherein the post translationally modified protein is phosphorylated, methylated, glycosyled, acetylated, ADP-ribosylated, nitrosylated, or farnesylated.

12. The peptide of claim 9, wherein the specific binding partner is an antibody or part of an antibody molecule.

13. The peptide of claim 9, wherein the peptide is derived from a phage display library.

14. The peptide of claim 9, further comprising an attached lanthanide, an attached dye, or an attached far red TR-FRET tracer.

15. A kit for detecting small molecular changes to a large molecule, comprising a peptide of claim 9.
